(19) United States
(12) Patent Application Publication
(10) Pub. No.: US 2008/0167690 A1
(43) Pub. Date: Jul. 10, 2008

(54) TREATMENT OF PERIPHERAL VASCULAR DISEASE BY BAROREFLEX ACTIVATION

(75) Inventors: Robert J. Cody, Minneapolis, MN (US); Robert S. Kieval, Medina, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CVRx, Inc., Minneapolis, MN (US)

(21) Appl. No.: 11/970,171

(22) Filed: Jan. 7, 2008

Related U.S. Application Data
(60) Provisional application No. 60/883,658, filed on Jan. 5, 2007.

(51) Int. Cl. A61N 1/00

(52) U.S. Cl. 607/2

(57) ABSTRACT

Peripheral vascular diseases, such as peripheral artery disease, are treated by activating a baroreflex response of a patient suffering from or at risk of these conditions. In the exemplary embodiments, the baroreflex response is activated using an implantable system comprising a controller and a device which activates the baroreceptor or baroreceptor nerve in order to initiate the baroreflex response.

### CROSS-REFERENCES TO RELATED APPLICATIONS

The present application claims the benefit of provisional U.S. Application No. 60/883,658 (Attorney Docket No. 021433-002100US), filed Jan. 5, 2007, the full disclosure of which is incorporated herein by reference.

This application is related to but does not claim the benefit of U.S. Pat. Nos. 6,522,926; and 6,616,624; 6,850,801; and 6,985,774, each of which are hereby fully incorporated by reference.

This application is also related to but does not claim the benefit of PCT Patent Application No. PCT/US01/30249, filed Sep. 27, 2001 (Attorney Docket No. 21433-000140PC) and the following U.S. patent application Nos., all of which are hereby incorporated fully by reference: 09/963,777 (Attorney Docket No. 21433-000120US), filed Sep. 26, 2001; 10/284,063 (Attorney Docket No. 21433-000150US), filed Oct. 29, 2002; 10/818,738 (Attorney Docket No. 21433-000160US), filed Apr. 5, 2004; 10/453,678 (Attorney Docket No. 21433-000210US), filed Jun. 2, 2003; 10/402,911 (Attorney Docket No. 21433-000410US), filed Mar. 27, 2003; 10/402,393 (Attorney Docket No. 21433-000420US), filed Mar. 27, 2003; 11/071,602 (Attorney Docket No. 21433-0011 US), filed Mar. 2, 2005; 11/168,231 (Attorney Docket No. 21433-001210US), filed Jun. 27, 2005; and 10/958,694 (Attorney Docket No. 021433-01610US), filed on Oct. 4, 2004.

### BACKGROUND OF THE INVENTION

1. Field of the Invention

The present invention relates generally to medical methods. More particularly, the present invention relates to methods for treating peripheral vascular disease (PVD) by activating a patient's baroreflex response.

Peripheral vascular disease (PVD) refers to all diseases of blood vessels outside the heart and brain. Often, the disease is manifested in the blood vessels of the arms and even more particularly of the legs. One form of PVD referred to as peripheral artery disease (PAD) results from the buildup of fatty deposits in the inner linings of the walls of arteries feeding the legs and other organs. In its early stages, PAD often results in cramping, pain or fatigue in the legs and buttocks during exercise. In severe cases, PVD and PAD can result in severely restricted blood flow, non-healing ulceration, and chronic gangrenous infection which sometimes necessitates leg amputation.

Less severe forms of PVD and PAD may be treated by lifestyle changes, particularly smoking cessation and control of other related conditions, such as diabetes, and high blood pressure. In more severe forms of the disease, conventional vascular interventions, such as angioplasty, atherectomy and stenting may be appropriate. Various medications have been tried with limited success.

Not all PVD is a secondary outcome of atherosclerosis. Vasculitis of diverse inflammatory, immunologic, or developmental etiologies may produce symptomatic or debilitating disease. An example of an intermittent vasospastic disorder is Raynaud's disease.

No one treatment for PVD and PAD has been found to be completely successful. Thus, it would be desirable to provide further alternative treatments which may be effective by themselves and/or in combination with prior treatment protocols. At least some of these objectives will be met by the inventions described herein below.

2. Description of the Background Art

Baroreflex stimulation for the control of hypertension is known. Various implantable systems for baroreflex activation are described in the patents in pending applications referred to in the Cross-Reference to Related Applications above. Baroreflex stimulation has been proposed for a number of other indications as well, including epilepsy control, heart failure treatment, and pain control. Treatment of these conditions is described in certain of the pending applications referred to above in the cross-referenced related applications.

### BRIEF SUMMARY OF THE INVENTION

The present invention provides methods and systems for activating a baroreflex response to treat patients suffering from or at risk of peripheral vascular disease (PVD), and in particular, peripheral arterial disease (PAD). It has been found that baroreflex activation increases blood flow through the peripheral arteries which in turn enhances perfusion of peripheral tissues, particularly tissues in the legs, to reduce the symptoms of PVD and PAD. By decreasing sympathetic nervous system outflow from the brain, baroreflex activation would improve local vasodilation. Current medications are limited by side effects, particularly in patients with chronic obstructive lung disease (eg beta-blockade). Surgical lumbar sympathectomy has been supplanted by “medical” sympathectomy of lumbar nerves. This is not uniformly successfule for the treatment of non-healing ulcers, and requires repeated procedures, which increase the serial risk of this therapy.

By improving blood flow, treatment of PAD and PVD with baroreflex activation would reduce patient symptoms at rest or associated with, for example, walking and more vigorous exercise. Improved blood flow would also be expected to improve healing of ischemic ulcers and other peripheral injuries. Raynaud's disease and other forms of vasculitis may be particularly amenable to systemic modulation of baroreflex circulatory control.

In one aspect of the present invention, a method for effecting a change in a baroreflex response of a patient to treat PVD and PAD comprises activating the baroreflex response of the patient with at least one baroreflex activation device. Optionally, the method may further involve implanting the baroreflex activation device in the patient. For example, in some embodiments, the device is implanted in an intravascular, extravascular and or intramural (within a vessel wall) location. In such embodiments, the method may also involve advancing the at least one baroreflex activation device through vasculature of the patient to a location for implantation. In one embodiment, for example, the device is advanced through venous vasculature of the patient. Optionally, in one embodiment, the device is advanced through a wall of the venous vasculature and is then implanted extravascularly on a wall of an artery. In various embodiments, any other suitable implantation locations and techniques may be employed.

In some embodiments, activating the baroreflex system involves activating a baroreceptor, one or more nerves coupled with a baroreceptor, a carotid sinus nerve, or some combination thereof. For example, in one embodiment, one or more baroreceptors are activated. Such baroreceptors, for example, may be located in the carotid sinus, aortic arch, heart, common carotid artery, subclavian artery, pulmonary artery, femoral artery, iliac artery, brachiocephalic artery and/or the like. In an alternative embodiment, such baroreceptors may be located in the inferior vena cava, superior vena cava, portal vein, jugular vein, subclavian vein, iliac vein, azygous vein, pulmonary vein, femoral vein and/or the like.

Activating the baroreflex may involve electrical activation, mechanical activation, thermal activation, chemical activation, some combination thereof, or any other suitable type of activation. In various embodiments, activation may be either continuous, pulsed or periodic. In one embodiment, activating the baroreflex system not only treats or reduces pain but also causes sedation of the patient.

In an embodiment, the level of therapy delivery is established by the physician or the patient.

In another embodiment, the system operates in a closed loop fashion with input from one or more sensors that are provided with the system that monitor one or more conditions of the patient.

In yet another embodiment, baroreflex activation therapy is used in conjunction with other medications and/or device therapies, such as the use of implantable drug pumps. Such additional therapies may provide separate, complementary or synergistic effects.

### BRIEF DESCRIPTION OF THE DRAWINGS

FIG. 1 is a schematic illustration of the upper torso of a human body showing the major arteries and veins and associated anatomy;

FIG. 2A is a cross sectional schematic illustration of a carotid sinus and baroreceptors within a vascular wall;

FIG. 2B is a schematic illustration of baroreceptors within a vascular wall and the baroreflex system.

FIG. 3 is a diagram of an implanted baroreflex activation system having an electrode which is disposed over the exterior of the carotid sinus for treatment of PVD and PAD according to the methods of the present invention. The system of FIG. 3 employs an external electrode.

FIG. 4 illustrates an intravascular electrode for stimulating a baroreceptor in the carotid sinus which could alternatively be employed with the system of FIG. 3.

### DETAILED DESCRIPTION OF THE INVENTION

Methods and systems according to the present invention are provided for treating patients suffering from or at risk of suffering from peripheral vascular disease (PVD), more particularly peripheral artery disease (PAD) in the extremities, particularly the legs. Patients suffering from PVD and PAD may be diagnosed based on medical history, ultrasound, X-ray angiography, and magnetic resonance imaging angiography (MRA). Once a patient is diagnosed, and other treatment options evaluated, patients may be selected for treatment by baroreflex activation based primarily on severity of the disease and on availability of other treatment options. Patients who have failed previous attempts at interventional procedures such as stednt placement, may be particularly logical candidates for the baroreflex activation therapies of the present invention.

Baroreflex activation is usually achieved by stimulation of one or more baroreceptors and/or baroreceptor nerves, both of which cause the patient's baroreflex response to dilate the arteries and enhance perfusion in peripheral tissues which may receive inadequate perfusion due to the peripheral vascular disease. The baroreflex receptor and/or nerve activation may be accomplished in a number of ways as described hereinbelow.

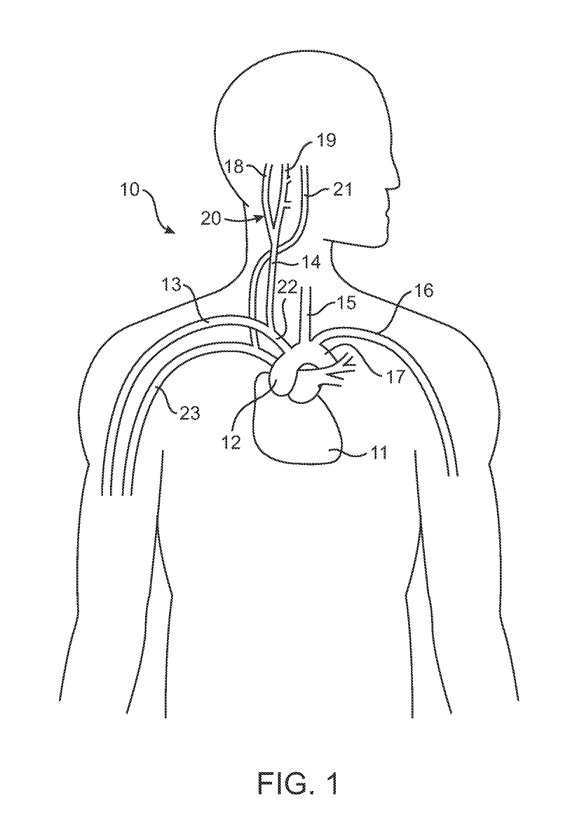

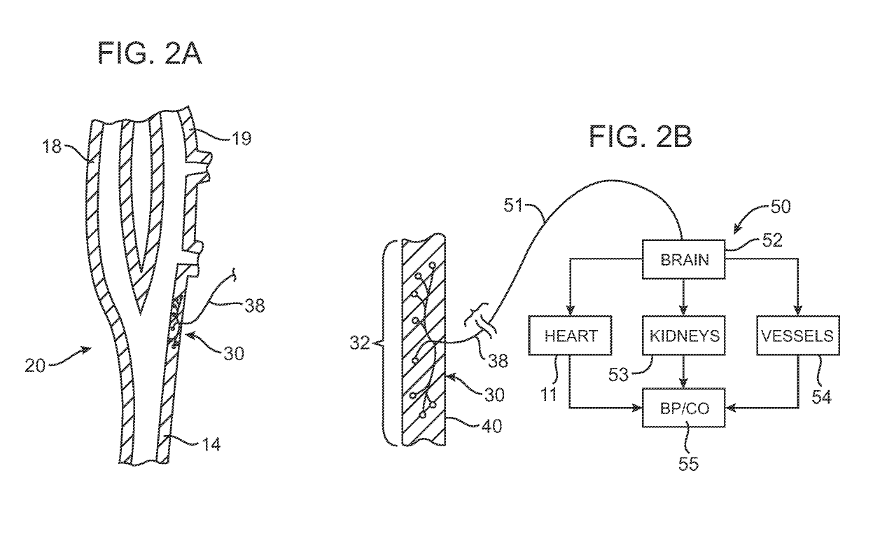

Referring now to FIGS. 1, 2A and 2B, within the arterial walls of the aortic arch 12, common carotid arteries 14/15 (near the right carotid sinus 20 and left carotid sinus), subclavian arteries 13/16 and brachiocephalic artery 22 there are baroreceptors 30. For example, as best seen in FIG. 2A, baroreceptors 30 reside within the vascular walls of the carotid sinus 20. Baroreceptors 30 are a type of stretch receptor used by the body to sense blood pressure. An increase in blood pressure causes the arterial wall to stretch, and a decrease in blood pressure causes the arterial wall to return to its original size. Such a cycle is repeated with each beat of the heart. The systemic lowering of the blood pressure has been found by the inventors herein to dilate the peripheral arteries and enhance tissue perfusion, thus providing relief to the symptoms of PVD and PAD. Baroreceptors 30 located in the right carotid sinus 20, the left carotid sinus and the aortic arch 12 play the most significant role in sensing blood pressure that affects baroreflex system 50, which is described in more detail with reference to FIG. 2B.

With reference now to FIG. 2B, a schematic illustration shows baroreceptors 30 disposed in a generic vascular wall 40 and a schematic flow chart of baroreflex system 50. Baroreceptors 30 are profusely distributed within the arterial walls 40 of the major arteries discussed previously, and generally form an arbor 32. The baroreceptor arbor 32 comprises a plurality of baroreceptors 30, each of which transmits baroreceptor signals to the brain 52 via nerve 38. Baroreceptors 30 are so profusely distributed and arborized within the vascular wall 40 that discrete baroreceptor arbors 32 are not readily discernable. To this end, baroreceptors 30 shown in FIG. 2B are primarily schematic for purposes of illustration.

In addition to baroreceptors, other nervous system tissues are capable of inducing baroreflex activation. For example, baroreflex activation may be achieved in various embodiments by activating one or more baroreceptors, one or more nerves coupled with one or more baroreceptors, a carotid sinus nerve or some combination thereof. Therefore, the phrase “baroreflex activation” generally refers to activation of the baroreflex system by any means, and is not limited to directly activating baroreceptor(s). Although the following description often focuses on baroreflex activation/stimulation and induction of baroreceptor signals, various embodiments of the present invention may alternatively achieve baroreflex activation by activating any other suitable tissue or structure. Thus, the terms “baroreflex activation device” and “baroreflex activation device” are used interchangeably in this application.

Baroreflex signals are used to activate a number of body systems which collectively may be referred to as baroreflex system 50. Baroreceptors 30 are connected to the brain 52 via the nervous system 51, which then activates a number of body systems, including the heart 11, kidneys 53, vessels 54, and other organs/tissues via neurohormonal activity. Although such activation of baroreflex system 50 has been the subject of other patent applications by the inventors of the present invention, the present invention results from the recognition that baroreflex activation enhances arterial perfusion of the peripheral tissue perfusion as a treatment for PVD and PAD.

Any suitable baroreflex activation device (or multiple devices) may be used in various embodiments. Examples of suitable baroreflex activation devices include, but are not limited to, those described in detail in U.S. Pat. Nos. 6,522,926; 6,616,624, 6,850,801; and 6,985,774, and U.S. patent application Ser. Nos. 09/963,777, 10/284,063, 10/453,678, 10/402,911, 10/402,393, 10/818,738, 10/958,694; 11/071,602; and 11/168,231, which were previously incorporated by reference. Any number or type of suitable baroreflex activation devices may be used, in accordance with various embodiments, and the activation device(s) may be placed in any suitable anatomical location. For further details regarding specific exemplary baroreflex activation devices, reference may be made to any of the patents or patent applications listed immediately above.

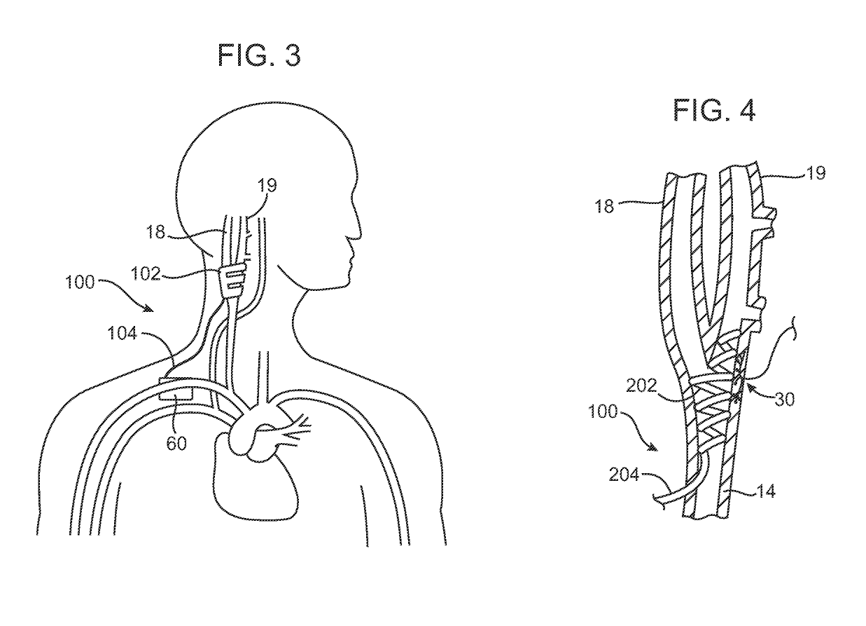

Referring now to FIGS. 3 and 4, a baroreflex activation system 100 comprises an implantable controller 60 and an external electrode 102 mounted over the carotid sinus at the junction of the common and carotid arteries 18 and 19. Suitable electrode and control systems are described in co-pending application Ser. Nos. 10/818,738 and 10/402,911, the full disclosures of which have been previously incorporated herein by reference. Alternative baroreflex activation systems could employ internal balloons 202 connected through an inflation line 204, as shown in FIG. 4. Still further internal and external baroreflex activation systems are described in U.S. Pat. No. 6,522,926, the full disclosure of which has been previously incorporated herein by reference.

Although the above description provides a complete and accurate representation of the invention, the present invention may be manifested in a variety of forms other than the specific embodiments described and contemplated herein. Accordingly, departures in form and detail may be made without departing from the scope and spirit of the present invention as described in the appended claims.

What is claimed is:

1. A method for treating peripheral vascular disease in a patient, said method comprising activating a baroreflex response of the patient with at least one baroreflex activation device.

2. A method as in claim 1, further comprising implanting the at least one baroreflex activation device.

3. A method as in claim 1, wherein activating is performed with an external device.

4. A method as in claim 1, wherein activating the baroreflex response comprises activating at least one of a baroreceptor, one or more nerves coupled with a baroreceptor, and a carotid sinus nerve.

5. A method as in claim 4, wherein at least one baroreceptor is activated.

6. A method as in claim 5, wherein the baroreceptor is located in at least one of a carotid sinus, aortic arch, heart, common carotid artery, subclavian artery, pulmonary artery, femoral artery, iliac artery and brachiocephalic artery.

7. A method as in claim 5, wherein the baroreceptor is located in at least one of an inferior vena cava, superior vena cava, portal vein, jugular vein, subclavian vein, iliac vein, azygous vein, pulmonary vein and femoral vein.

8. A method as in claim 1, wherein activating comprises at least one of electrical activation, mechanical activation, thermal activation and chemical activation.

9. A method as in claim 1, wherein activating comprises at least one of continuous activation, pulsed activation and periodic activation.

10. A method for improving blood flow in a patient suffering from PAD or PVD, comprising activating a baroreflex response of the patient with at least one baroreflex activation device.

11. A method for reducing symptoms associated with PVD or PAD, comprising activating a baroreflex response of the patient with at least one baroreflex activation device.

12. The method of claim 11, whereby the symptoms are experienced at rest.

13. The method of claim 11, wherein the symptoms are experienced with walking.

14. The method of claim 11, wherein the symptoms are experienced with vigorous exercise.

15. A method of improving healing of ischemic ulcers in a patient suffering from PAD, comprising activating a baroreflex response of the patient with at least one baroreflex activation device.

16. A method of treating Raynaud's disease, comprising activating a baroreflex response of the patient with at least one baroreflex activation device.

17. A method of treating vasculitis in a patient, comprising activating a baroreflex response of the patient with at least one baroreflex activation device.

18. A method for treating PAD/PVD, comprising baroreflex activation in combination with one or more additional medical therapies.

19. A method as in claim 18, wherein the one or more additional therapies are selected from the group consisting of drug therapies and device therapies.
